(19) United States
(12) Patent Application Publication
(10) Pub. No.: US 2020/0154725 A1
(43) Pub. Date: May 21, 2020

(54) Method for Producing Healthy THC Cooking Oil and Budder

(71) Applicant: Jerome Williams, Orlando, FL (US)

(72) Inventor: Jerome Williams, Orlando, FL (US)

(21) Appl. No.: 16/452,116

(22) Filed: Jun. 25, 2019

Related U.S. Application Data
(60) Provisional application No. 62/769,419, filed on Nov. 19, 2018.

(51) Int. Cl. A23D 9/007; A61K 36/185; A61K 9/00; A61K 31/352; A23D 9/04

(52) U.S. Cl. CPC A23D 9/007; A61K 36/185; A23D 9/04; A61K 31/352; A61K 9/0056

(57) ABSTRACT

A method for producing cannabinoid cooking oil and budder uses a mixture of cannabinoid material and natural oils to produce a consumable spread. The method used a quantity of cannabinoid substance, and a quantity of foaming substance. The foaming substance is a natural oil that can absorb and blend with the cannabinoid substance. The method begins by continuously agitating the cannabinoid substance with the foaming substance until a paste mixture is formed. The method continues by cooling the paste mixture until a solidified mixture is formed. The method concludes by thawing the solidified mixture to yield a tetrahydrocannabinol (THC) infused spreadable paste. Thus, an edible mixture is produced. Some versions of the method feature a flavoring substance that is added to the cannabinoid and foaming substance mixture. Thus, improving flavor.

The current application claims a priority to the U.S. Provisional Patent application Ser. No. 62/769,419 filed on Nov. 19, 2018.

### FIELD OF THE INVENTION

The present invention relates generally to edible spread and other related products. More specifically, the present invention is a method of producing a cannabinoid spread.

### BACKGROUND OF THE INVENTION

In present society, there has been an increasing acceptance of cannabis-based products and cannabis products as more governments roll back prohibition of such products. A popular product that would fall under cannabis-based products or cannabis products is commonly referred to as marijuana butter. Although marijuana butter has been around for quite some time, there has not been much development in the improvement of marijuana butter. One of the most major setbacks of marijuana butter can be its aid in the risk of developing unhealthy side-effects in consumers of marijuana butter, such as higher cholesterol and an increase in risk of developing heart disease; much like with non-cannabis-based butter and similar products. However, through how the marijuana butter is produced, there can be ways in which these health risk factors can be mitigated or avoided.

An objective of the present invention is to provide users with a method that can produce a more healthier marijuana butter or cannabinoid spread, which can be consumed. The present invention intends to provide users with a method that is more simplified and easier to perform in order to achieve the desired cannabinoid spread of the present invention. The present invention intends to provide users with a method that can achieve the desired cannabinoid spread of the present invention more quickly.

### BRIEF DESCRIPTION OF THE DRAWINGS

FIG. 1 is a flowchart depicting the overall process of the present invention.

FIG. 2 is a flowchart depicting the steps necessary to adequately infusing a foaming substance with a cannabinoid substance.

FIG. 3 is a flowchart depicting the steps necessary to yield a solidified mixture from a paste mixture.

FIG. 4 is a flowchart depicting the steps necessary to yield a THC infused spreadable paste.

FIG. 5 is a flowchart depicting the steps necessary to infuse a flavor enhancer into the paste mixture.

### DETAIL DESCRIPTIONS OF THE INVENTION

All illustrations of the drawings are for the purpose of describing selected versions of the present invention and are not intended to limit the scope of the present invention.

The present invention generally relates to methods for creating edible spread infused with cannabis. Specifically, the present invention is a method for producing cannabinoid spread that is significantly healthier than traditional marijuana butter. The present invention may be used as a cannabis cooking oil as a healthier alternative to standard cooking oils.

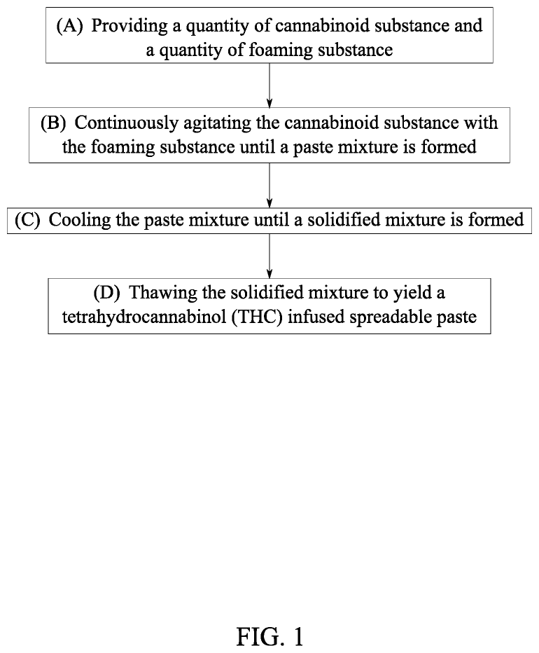

Referring to FIG. 1, the ingredients for the present invention include a quantity of cannabinoid substance, a quantity of foaming substance, and a quantity of flavor enhancer (Step A). The present invention delineates a method which utilizes the cannabinoid substance, the foaming substance, and the flavor enhancer to create a tetrahydrocannabinol (THC) infused spreadable paste. The cannabinoid substance is a chemical compound that acts on cannabinoid receptors in cells that alter neurotransmitter release in the brain. The cannabinoid substance is any derived substance from cannabis flowering plant in any form or fashion. The cannabinoid substance provides medical benefits to the consumer including, but not limited to, treating chronic pain, aiding with alcohol or opioid dependencies, relieving depression, alleviating stress/anxiety, decreasing nausea and vomiting, and promoting a healthy appetite to name a few non-limiting examples. In the preferred embodiment of the present invention, the cannabinoid substance includes tetrahydrocannabinol (THC), the primary psychoactive constituent in cannabis. Additionally, the cannabinoid substance is utilized in the liquid form, an oil specifically. The foaming substance facilitates the formation of foam/suds during the mixing process to yield a paste-like form. Type of substances that may be used as the foaming substance include, but are not limited to, honey oil, olive oil, coconut oil, avocado oil, natural oil, egg yolk, butter, Crisco, butter, flaxseed oil, and other similar foaming agents or emulsifiers. The paste-like form allows the resulting THC infused spreadable paste to be sold, packaged, and used as a spread; i.e. a food item to be put over the surface of bread, crackers, and other similar food items. The flavor enhancer brings out the existing flavor within the THC infused spreadable paste. Additionally, the flavor enhancer may be used to alter the taste/flavor of the THC infused spreadable paste. Type of substances that may be used as the flavor enhancer includes, but is not limited to vanilla extract, vegetable oil, olive oil, natural oil, coconut oil, peanut oil, vegetable oil, flaxseed oil, and any other edible enhancers utilized in the culinary arts.

Referring to FIG. 1, in the simplest embodiment of the present invention, the flavor enhancer is not utilized. In general, the method for creating the THC infused spreadable paste first begins with continuously agitating the cannabinoid substance with the foaming substance until a paste mixture is formed (Step B). Continuous agitation evenly mixes the cannabinoid substance and the foaming substance while simultaneously forming small bubbles throughout, i.e. frothing. Next, the paste mixture is cooled until a solidified mixture is formed (Step C). The solidified mixture is a solid state of the mixed cannabinoid substance and the foaming substance that ensures the homogenous degree achieved in Step B is maintained semi-permanent. Finally, the solidified mixture is thawed until the solidified mixture yields a THC infused spreadable paste (Step D). The THC infused spreadable paste is a butter-like consistency, i.e. solid when chilled and liquid when warmed. The THC infused spreadable paste may be used as a spread for bread products and as a condiment for cooking processes.

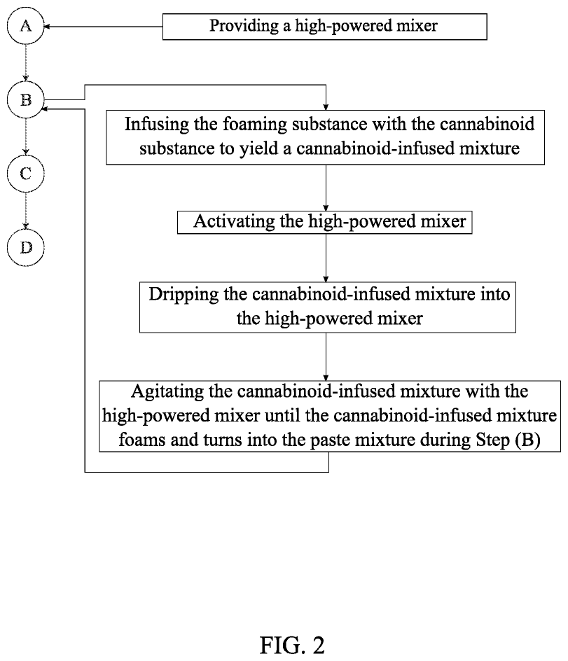

Referring to FIG. 2, Step B, is specifically implemented with a high-powered mixer, i.e. a stand mixer; although other devices and mechanisms may be utilized in alternative embodiments. Step B begins with infusing the foaming substance with the cannabinoid substance to yield a cannabinoid-infused mixture. This is essentially pouring the foaming substance and the cannabinoid substance into a single container, such as a bowl. Next, the high-powered mixer is activated, and the cannabinoid-infused mixture is slowly dripped into the high-powered mixer, specifically a mixing bowl of the high-powered mixer. As the cannabinoid-infused mixture is dripped into the high-powered mixer, small portions the cannabinoid-infused mixture are agitated by the high-powered mixer at a time until the cannabinoid-infused mixtures fully foams and turns into the paste mixture. This process ensures that the paste mixture is fully homogenous.

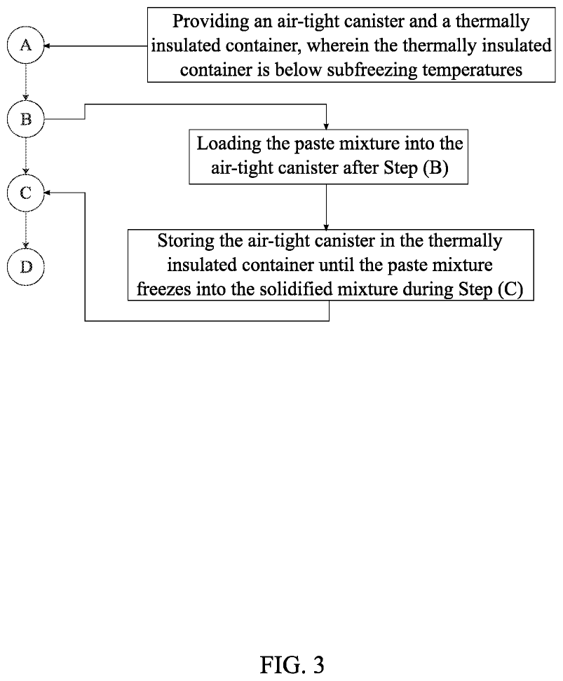

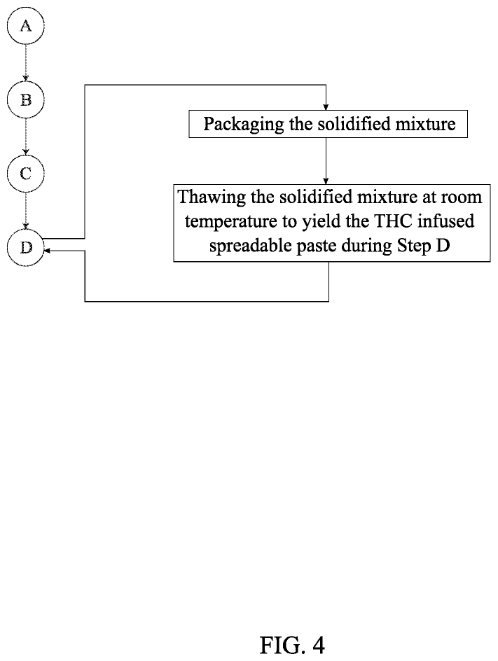

Referring to FIG. 3, during Step C, the paste mixture is frozen specifically through the use of an air-tight canister and a thermally insulated container. After Step B, the paste mixture is loaded into air-tight canister in order to seal the paste mixture and prevent any debris or other unwanted impurities from contaminating the paste mixture during the cooling process. Next, the air-tight canister is stored in the thermally insulated container until the paste mixture freezes into the solidified mixture during Step C; wherein the thermally insulated container is a freezer that has an interior temperature below subfreezing. The amount of time necessary to solidify the contents within the air-tight canister is subject to change depending on batch size, interior temperature, and other relevant factors. Once the solidified mixture is formed, the solidified mixture is packaged into an at least one container to be sold, distributed, stored, or otherwise transported. In order to utilize the final product of the present invention, the solidified mixture is thawed to yield the THC infused spreadable paste during Step D, similar to butter, as seen in FIG. 4.

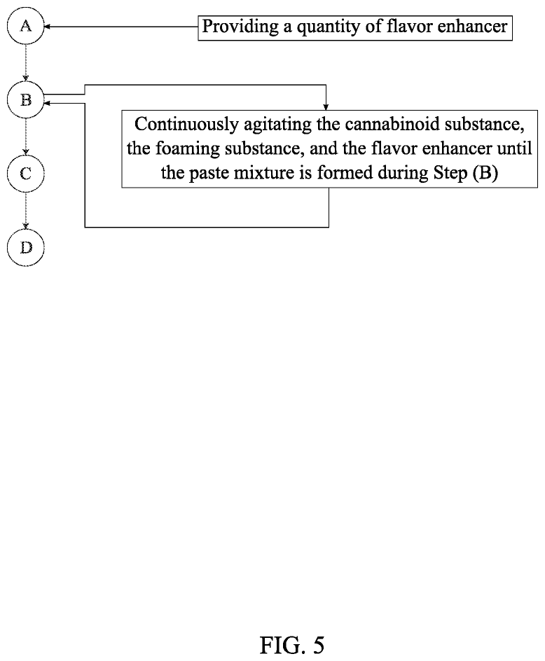

Referring to FIG. 5, in one embodiment of the present invention, the flavor enhancer is used to accentuate and or alter the taste of the THC infused spreadable paste with the flavor enhancer. For this, Step B is modified to include the flavor enhancer. Specifically, during Step B, the cannabinoid substance, the foaming substance, and the flavor enhancer are continuously agitated until the paste mixture is formed.

While the relative quantity of the ingredients used for the present invention is subject to change, a preferred weight percentage is as follows; the quantity of cannabinoid substance being about 40 percentage by weight (wt. %) to about 49 wt. % of the THC infused spreadable paste; the quantity of foaming substance being about 40 wt. % to about 49 wt. % of the THC infused spreadable paste; and, the quantity of the flavor enhancer being about 2 wt. % to about 20 wt. % of the THC infused spreadable paste. Although, in alternative embodiments of the present invention, the relative quantity between the cannabinoid substance, the flavor enhancer, and the foaming substance may differ to meet the needs and preferences of the consumer.

In one embodiment, the present invention includes a relatively high amount of THC. In this embodiment, the quantity of flavor enhancer is about 2 wt. % of the THC infused spreadable paste, the quantity of foaming substance is about 18 wt. % of the THC infused spreadable paste, and the quantity of cannabinoid substance is about 80 wt. % of the THC infused spreadable paste. In another embodiment of the present invention, the present invention includes a medium amount of THC. In this embodiment, the quantity of flavor enhancer is about 20 wt. % of the THC infused spreadable paste, the quantity of foaming substance is about 40 wt. % of the THC infused spreadable paste, and the quantity of cannabinoid substance is about 40 wt. % of the THC infused spreadable paste. In yet another embodiment, the present invention includes a relative low amount of THC. In this embodiment, the quantity of flavor enhancer is about 2 wt. % of the THC infused spreadable paste, the quantity of foaming substance is about 80 wt. % of the THC infused spreadable paste, and the quantity of cannabinoid substance is about 18 wt. % of the THC infused spreadable paste.

Although the invention has been explained in relation to its preferred embodiment, it is to be understood that many other possible modifications and variations can be made without departing from the spirit and scope of the invention as hereinafter claimed.

What is claimed is:

1. A method for producing cannabinoid cooking oil and budder comprising the steps of:
(A) providing a quantity of cannabinoid substance and a quantity of foaming substance;
(B) continuously agitating the cannabinoid substance with the foaming substance until a paste mixture is formed;
(C) cooling the paste mixture until a solidified mixture is formed; and
(D) thawing the solidified mixture to yield a tetrahydrocannabinol (THC) infused spreadable paste.

2. The method for producing cannabinoid cooking oil and budder as claimed in claim 1 comprising the steps of:
providing a high-powered mixer;
infusing the foaming substance with the cannabinoid substance to yield a cannabinoid-infused mixture;
activating the high-powered mixer;
dripping the cannabinoid-infused mixture into the high-powered mixer; and
agitating the cannabinoid-infused mixture with the high-powered mixer until the cannabinoid-infused mixture foams and turns into the paste mixture during Step (B).

3. The method for producing cannabinoid cooking oil and budder as claimed in claim 1 comprising the steps of:
providing an air-tight canister and a thermally insulated container, wherein the thermally insulated container is below subfreezing temperatures;
loading the paste mixture into the air-tight canister after Step (B); and
storing the air-tight canister in the thermally insulated container until the paste mixture freezes into the solidified mixture during Step (C).

4. The method for producing cannabinoid cooking oil and budder as claimed in claim 1 comprising the steps of:
packaging the solidified mixture; and
thawing the solidified mixture at room temperature to yield the THC infused spreadable paste during Step (D).

5. The method for producing cannabinoid cooking oil and budder as claimed in claim 1 comprising the steps of:
providing a quantity of flavor enhancer; and
continuously agitating the cannabinoid substance, the foaming substance, and the flavor enhancer until the paste mixture is formed during Step (B).

6. The method for producing cannabinoid cooking oil and budder as claimed in claim 5, wherein the flavor enhancer is selected from the group consisting of vanilla extract, coconut oil, olive oil, natural oil, peanut oil, flaxseed oil, and vegetable oil.

7. The method for producing cannabinoid cooking oil and budder as claimed in claim 1, wherein the foaming substance is selected from the group consisting of honey oil, egg yolk, butter, coconut oil, olive oil, avocado oil, flaxseed oil, and natural oil;

8. The method for producing cannabinoid cooking oil and budder as claimed in claim 1, wherein the quantity of cannabinoid substance is about 40 percentage by weight (wt. %) to about 49 wt. % of the THC infused spreadable paste;

9. The method for producing cannabinoid cooking oil and budder as claimed in claim 1, wherein the quantity of foaming substance is about 40 wt. % to about 49 wt. % of the THC infused spreadable paste;

10. The method for producing cannabinoid cooking oil and budder as claimed in claim 5, wherein the quantity of flavor enhancer is about 2 wt. % to about 20 wt. % of the THC infused spreadable paste;

11. The method for producing cannabinoid cooking oil and budder as claimed in claim 5, wherein the quantity of flavor enhancer is about 2 wt. % of the THC infused spreadable paste, the quantity of foaming substance is about 18 wt. % of the THC infused spreadable paste, and the quantity of cannabinoid substance is about 80 wt. % of the THC infused spreadable paste.

12. The method for producing cannabinoid cooking oil and budder as claimed in claim 5, wherein the quantity of flavor enhancer is about 20 wt. % of the THC infused spreadable paste, the quantity of foaming substance is about 40 wt. % of the THC infused spreadable paste, and the quantity of cannabinoid substance is about 40 wt. % of the THC infused spreadable paste.

13. The method for producing cannabinoid cooking oil and budder as claimed in claim 5, wherein the quantity of flavor enhancer is about 2 wt. % of the THC infused spreadable paste, the quantity of foaming substance is about 80 wt. % of the THC infused spreadable paste, and the quantity of cannabinoid substance is about 18 wt. % of the THC infused spreadable paste.
